(19) United States
(12) Patent Application Publication
(10) Pub. No.: US 2026/0232427 A1
(43) Pub. Date: Aug. 13, 2026

(54) Non-Suture Soft Tissue Anchor Device

(71) Applicants: Thomas Zink, San Antonio, TX (US); Christopher Hyer, Columbug, OH (US); Thomas S. Roukis, Winter Garden, FL (US)

(72) Inventors: Thomas Zink, San Antonio, TX (US); Christopher Hyer, Columbug, OH (US); Thomas S. Roukis, Winter Garden, FL (US)

(21) Appl. No.: 19/049,101

(22) Filed: Feb. 10, 2025

(51) Int. Cl. A61F 2/08; A61B 17/86

(52) U.S. Cl. CPC A61F 2/0811; A61B 17/866; A61F 2002/0829; A61F 2002/0864; A61F 2002/0888

(57) ABSTRACT

A non-suture soft tissue anchor device is disclosed, which is used in orthopedic surgical cases for attaching tendons and ligaments (i.e., soft tissue) to bone. The non-suture soft tissue anchor device comprises a body component configured in an oval shape. Further, the body component comprises at least two through-holes, with one through-hole positioned on each opposing narrow end of the body component. The through-holes are sized and shaped to accept a bone screw to secure the device to the bone. Further, the body component comprises two or more slots to accept the soft tissue. Specifically, the soft tissue is weaved through the slots like a belt buckle to lock the soft tissue in place. The soft tissue is locked in place at a set tension. The slots define a moveable lock bar, which further locks the soft tissue between the bone and the device.

### FIELD OF THE INVENTION

The present invention relates generally to a medical device for use in the field of orthopaedic surgery and related fields. More particularly, the present invention relates to a soft tissue anchor for attaching tendons and ligaments to bone. This implant does not violate the structural integrity of the soft tissue and allows it to be tensioned once in place on the bone. Accordingly, the present disclosure makes specific reference thereto. Nonetheless, it is to be appreciated that aspects of the present invention are also equally applicable to other like applications, devices and methods of manufacture.

### BACKGROUND

Generally, with respect to deformity corrections wherein tendons need to be moved around and repositioned, there is a need in orthopedic surgical cases to attach soft tissue (i.e., tendons and ligaments) to bone to retain the tendons in place, once repositioned. However, the soft tissue is not as rigid as bone and can re-tear if any defect is present within the soft tissue. Defects can be small tears or holes in the soft tissue, these defects act as a stress riser and can structurally weaken the soft tissue. Also, once the soft tissue is position, the soft tissue must be tensioned before it is attached. Thus, the structural integrity of the soft tissue must not be violated and it must be able to be tensioned, once in place on the bone.

Most existing technology uses suture anchors or interference screws to attach soft tissue to bone. However, both suture anchors and interference screws use sutures to attach or to pull the soft tissue through or to a bone. Further, to attach the suture to the soft tissue, use of a needle to pierce the soft tissue is used, thus creating a defect in the soft tissue. Another existing technology to attach soft tissue to bone is a barbed button or plate. These technologies use a washer-like plate that has barbs on the back side to pierce the soft tissue and the bone under the tissue. The button or plate is then screwed through the tissue or next to the tissue into the bone. This technology also violates the integrity of the soft tissue causing it to lose strength and allows for failures.

Furthermore, there are a variety of deformities that require tendons to be moved around and repositioned. These tendons and/or ligaments (i.e., soft tissue), must then be tensioned and secured to bone, once in the new position. However, the soft tissue must maintain its structural integrity and be able to be tensioned once in place on the bone in order to correct the defect.

Accordingly, it would be desirable to provide an implant that does not violate the structural integrity of the soft tissue and allows it to be tensioned once in place on the bone. Further, it would be desirable to attach tendons and ligaments (i.e., soft tissue) to bone in a secure means and at the desired tension required.

The subject matter disclosed and claimed herein, in one embodiment thereof, comprises a non-suture soft tissue anchor device. The non-suture soft tissue anchor device is used in orthopedic surgical cases for attaching tendons and ligaments (i.e., soft tissue) to bone. The non-suture soft tissue anchor device comprises a body component configured in an oval shape, such that a middle part tapers to opposing narrow ends to form the oval shape. Further, the body component comprises a top surface, a bottom surface, and a curved sidewall which runs along the perimeter, defining the oval shape. The body component comprises at least two through-holes, with one through-hole positioned on each opposing narrow end of the body component. The through-holes are sized and shaped to accept a bone screw to secure the device to the bone.

Further, the body component comprises two or more slots to accept the soft tissue. Specifically, the soft tissue is weaved through the slots like a belt buckle to lock the soft tissue in place. The soft tissue is locked in place at a set tension. The slots are configured in an oval shape and span the length of the middle part. The slots define a moveable lock bar, which further locks the soft tissue between the bone and the device. The moveable lock bar is configured in a rectangular shape and sits lower than the bottom surface of the body component and contacts the bone when the device is in position. Further, the bottom surface of the body component comprises a plurality of ridges to further help secure the device to the bone. The plurality of ridges contact the bone when the device is in position. The plurality of ridges act to mitigate migration of the soft tissue once in position, securing the device in place.

Furthermore, the device is intended to be machined or molded from PEEK or PEEK enhanced with hydroxyapatite to promote boney on-growth.

In use, a soft tissue anchor would be placed in a desired position on the bone that the soft tissue needs to be attached to. Screw holes are then drilled through the through-holes in the device and screws are placed through the holes and into the bone. The soft tissue is then weaved through the slots in the device. Once the soft tissue is completely through the slots, the surgeon tensions the soft tissue to a desired tension and secures the device to the bone.

In this manner, the non-suture soft tissue anchor device of the present invention accomplishes all of the forgoing objectives and provides users with a device that secures soft tissue to bone at a desired tension. The device does not violate the structural integrity of the soft tissue and allows it to be tensioned once in place on the bone.

### SUMMARY OF THE INVENTION

The following presents a simplified summary in order to provide a basic understanding of some aspects of the disclosed innovation. This summary is not an extensive overview, and it is not intended to identify key or critical elements or to delineate the scope thereof. Its sole purpose is to present some concepts in a simplified form as a prelude to the more detailed description that is presented later.

The subject matter disclosed and claimed herein, in one embodiment thereof, comprises a non-suture soft tissue anchor device. The non-suture soft tissue anchor device is used in orthopedic surgical cases for attaching tendons and ligaments (i.e., soft tissue) to bone. The non-suture soft tissue anchor device comprises a body component configured in an oval shape. The body component comprises at least two through-holes, sized to accept a bone screw to secure the device to the bone. Further, the body component comprises two or more slots to accept the soft tissue. Specifically, the soft tissue is weaved through the slots like a belt buckle to lock the soft tissue in place. The slots define a moveable lock bar, which further locks the soft tissue between the bone and the device.

In one embodiment, the non-suture soft tissue anchor device comprises a body component configured in an oval shape, such that a middle part tapers to opposing narrow ends to form the oval shape. The body component can be configured in multiple sizes depending on the size of the tendon to be secured. Further, any suitable shape as is known in the art can be utilized for the body component, as well, such as square, rectangular, circular, etc.

In one embodiment, the body component comprises a top surface, a bottom surface, and a curved sidewall which runs along the perimeter, defining the oval shape. Further, the top surface and the bottom surface share the curved sidewall extending therebetween. The bottom surface contacts the bone and the top surface is positioned away from the bone, when the device is in position.

In one embodiment, the body component comprises at least two through-holes, with one through-hole positioned on each opposing narrow end of the body component. The through-holes are sized and shaped to accept a bone screw to secure the device to the bone. The bone screws can be any suitable bone screws as is known in the art including locking screws.

In one embodiment, the body component is secured without bone screws. In this embodiment, a user would insert wires, pins, or staples into the two through-holes, to secure the device to the bone.

In one embodiment, the device can be machined, molded or 3D printed. Further, the device and its components may comprise any synthetic or natural homogenous material suitable for implantation into bone, including any one or more of collagen, human or animal allograft, silicone, bioglass, collagen, PEEK, polyethylene, titanium, stainless steel, cobalt chrome, and the like, but is typically manufactured of PEEK or PEEK enhanced with hydroxyapatite.

In one embodiment, the non-suture tissue anchor device may be implemented with a range of sizes that facilitate securing the soft tissue to bone in various locations in the human body, such as by way of non-limiting example, the dorsal aspect of the foot, the medial aspect of the foot, the lateral aspect of the foot, the anterior portion of the calcaneus, as needed, as well as multiple portions of the hand. It is contemplated, however, that the overall size of the device is to be selected according to the particular soft tissue that is to be attached. Thus, the device can be scaled up or down based on the size of the soft tissue and the position of the device on the cortical bone; and the device acts to secure the soft tissue to the bone without the use of multiple devices.

In one embodiment, the device possesses a thickness that extends from the bottom surface to the top surface. Generally, the thickness is substantially 1-3 millimeters (mm). It is contemplated, however, that the thickness may be varied according to placement in the foot and/or ankle and hand and/or wrist, and thus the device may be implemented with a wide variety of thicknesses, without limitation. The device can also be used in non-spine orthopaedic applications.

In one embodiment, when the non-suture soft tissue anchor device is implanted to secure the soft tissue to the cortical surface of the bone, the top surface of the device is disposed slightly above the cortical bone's surface. In general, the top surface includes a shape configured to approximate the cortical bone's surface. In some embodiments, the shape of the top surface includes a convex curvature that approximates the curvature of the cortical surface of the bone. In one embodiment, the top surface may have a flat curvature depending on the curvature of the cortical bone on which it is secured.

In one embodiment, the body component comprises two or more slots to accept the soft tissue during use of the device. Specifically, the soft tissue is weaved through the slots like a belt buckle to lock the soft tissue in place. The soft tissue is locked in place at a set tension. Thus, the device provides the ability to lock the soft tissue at a specific tension without violating the structural integrity of the soft tissue. The slots are configured in an oval shape and span the length of the middle part. However, the slots can be configured in any suitable shape and size as is known in the art, as long as they are of the appropriate length and width to accept soft tissue. After the soft tissue is fed through the slots, the surgeon/user can tension the soft tissue to a desired tension. If the initial tension is later found to be inadequate, the surgeon/user can pull the soft tissue and/or reposition the soft tissue within the slots. Thus, the device allows for the re-tensioning of soft tissue, multiple times, if needed.

In one embodiment, the slots define a moveable lock bar, which further locks the soft tissue between the bone and the device. The moveable lock bar is configured in a rectangular shape and sits lower than the bottom surface of the body component and contacts the bone when the device is in position. The moveable lock bar is positioned between the two slots. In use, the soft tissue is passed up through a first slot and over the center moveable lock bar and then threaded through the second (or opposing) slot and pulled out. Once the soft tissue is completely threaded through the slots and moveable lock bar, the surgeon/user can tension the soft tissue to a desired tension. The screws can then be tightened down to pull the device in contact with the bone. In tightening the device to the bone, the center moveable lock bar will then deform and be pushed away from the bone and will further act to lock the soft tissue into the device. Thus, the device allows the surgeon to tension the soft tissue and lock the tissue at a desired tension, without having to move or relocate the body component secured to the bone.

In one embodiment, the bottom surface of the body component comprises a plurality of ridges to further help secure the device to the bone. The plurality of ridges contact the bone and/or the soft tissue when the device is in position. The plurality of ridges act to mitigate migration of the soft tissue once in position, securing the device in place. Specifically, the ridges are triangular with the point pushing down toward the bone. Further, the ridges are of any suitable length, as long as the length enables pushing of the soft tissue to the bone without causing unnecessary damage to the soft tissue.

In one embodiment, tendons typically glide within synovial sheaths. The sheaths often contain synovial fluid. Synovial fluid lubricates the tendons, promoting a smooth glide between the tendons and the sheaths. The synovial fluid is also the primary source of nutrition for the fibrocyte cells of the tendon. The non-suture soft tissue anchor device does not act to cover the soft tissue during use, and thus allows synovial fluid to reach the tendon and prevents necrosis of the tendon, while speeding the healing process.

In one embodiment, the non-suture soft tissue anchor system for securing soft tissue to bone according to the present disclosure includes the soft tissue anchor device, a drill, a screw inserter, and bone screws. As will be appreciated, the non-suture soft tissue anchor system comprises instruments necessary for securing soft tissue to bone by way of surgery. The sizes of the instruments comprising the anchor system will depend upon the size of the particular device to be utilized. It is envisioned, therefore, that a surgeon may select the device and a correspondingly sized instrument based on the location and size of the soft tissue to be secured. The instruments comprising the soft tissue anchor system are not to be limited to the specific instruments, or the sizes and shapes of the instruments disclosed.

In operation, first, the soft tissue anchor device is placed in the desired position on the cortical bone that is where the soft tissue is needed to be attached to. Next, screw holes are then drilled through the opposing through-holes of the body component via the drill. Then, screws are placed through the drill holes and into the bone and tightened via the screw inserter. Typically, the screws are torqued into the bone but not sealed completely to the bone. Thus, there is a gap left between the body component and the bone. Once the body component is in position, the soft tissue is woven through the slots to be secured. Specifically, the soft tissue is placed under the bottom surface of the body component, the soft tissue is then passed up through a first slot and over the center moveable lock bar. After the soft tissue is passed over the center moveable lock bar, the soft tissue is then threaded through the second (or opposing) slot and pulled out under the opposing bottom surface of the body component.

In one embodiment, once the soft tissue is completely threaded through the slots and moveable lock bar, the surgeon/user can tension the soft tissue to a desired tension. The surgeon/user can pull back and forth on the soft tissue to achieve the desired tension. Once the desired tension is reached, the screws are then tightened down via the screw inserter to pull the soft tissue anchor device in contact with the cortical bone. In tightening the device to the bone, the center moveable lock bar will then deform and be pushed away from the bone and will further act to lock the soft tissue into the device. Thus, the device allows the surgeon to tension the soft tissue, re-tension the soft tissue, and finally lock the soft tissue at a desired tension, all without having to move or relocate the body component secured to the bone.

In general, it is contemplated that the non-suture soft tissue anchor system is to be suitably sterilized for surgeries and packaged into sterilized containers.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIGS. 1A-AC illustrate a perspective view of the non-suture soft tissue anchor device of the present invention in accordance with the disclosed architecture;

FIG. 2 illustrates a perspective view of the non-suture soft tissue anchor device of the present invention in use in accordance with the disclosed architecture;

FIG. 3 illustrates a sectional view of the non-suture soft tissue anchor device of the present invention in use in accordance with the disclosed architecture; and

FIGS. 4A-4D illustrate a perspective view of the non-suture soft tissue anchor device of the present invention before and after the soft tissue is locked into place in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

In this application, the words proximal, distal, anterior or plantar, posterior or dorsal, medial and lateral are defined by their standard usage for indicating a particular part or portion of a bone, or directional terms of reference, according to the relative disposition of the natural bone. For example, “proximal” means the portion of a bone nearest the torso, while “distal” indicates the portion of the bone farthest from the torso. As an example of directional usage of the terms, “anterior” refers to a direction towards the front side of the body, “posterior” refers to a direction towards the back side of the body, “medial” refers to a direction towards the midline of the body and “lateral” refers to a direction towards the sides or away from the midline of the body. Further, specifically in regard to the foot, the term “dorsal” refers to the top of the foot and the term “plantar” refers the bottom of the foot.

Similarly, positions or directions may be used herein with reference to anatomical structures or surfaces. For example, as the current devices, instrumentation and methods are described herein with reference to use with the bones of the foot, the bones of the foot, ankle and lower leg may be used to describe the surfaces, positions, directions or orientations of the devices, instrumentation and methods. Further, the devices, instrumentation and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the devices, instrumentation and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the devices, instrumentation and methods, and the aspects, components, features and the like thereof, described herein with respect to the right foot may be mirrored so that they likewise function with the left foot. Further, the devices, instrumentation and methods, and the aspects, components, features and the like thereof, disclosed herein are described with respect to the foot for brevity purposes, but it should be understood that the devices, instrumentation, and methods may be used with other bones of the body having similar structures, for example the upper extremity, and more specifically, with the bones of the wrist, hand, and arm.

As noted above, there is a long felt need in the art for devices, instrumentation, and methods of securing soft tissue to a bone via the non-suture soft tissue anchor device. Specifically, devices, instrumentation and methods used to secure soft tissue to bone without violating structural integrity of the soft tissue.

The present invention, in one exemplary embodiment, is a novel non-suture soft tissue anchor system. The system comprises a non-suture soft tissue anchor device which secures soft tissue to bone. The non-suture soft tissue anchor device comprises a body component with slots and a center lock bar. The instrumentation includes a drill, a screw inserter, and screws for securing the body component to the bone, among other instruments.

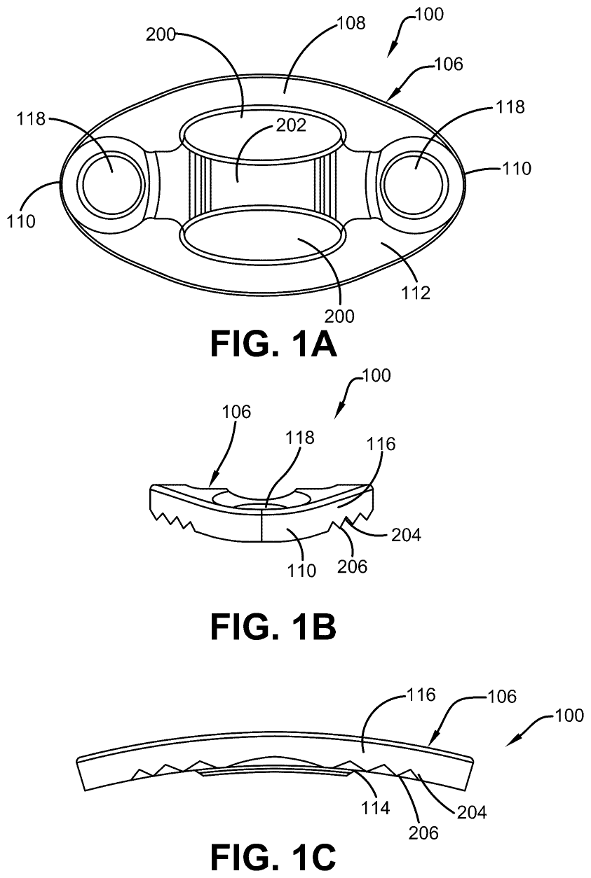

Referring initially to the drawings, FIGS. 1-4 illustrate multiple views of the non-suture soft tissue anchor device 100 of the present invention. In the present embodiment, the non-suture soft tissue anchor device 100 is an improved non-suture soft tissue anchor device 100 that secures soft tissue 102 to bone 104. The device 100 is especially designed to allow any doctor, surgeon, etc., or any other suitable user as is known in the art, to preserve the structural integrity of the soft tissue 102 and allows it to be tensioned once in place on the bone 104. More specifically, the non-suture soft tissue anchor system 400 comprises the non-suture soft tissue anchor device 100 and instrumentation for securing the device 100 to bone 104.

As shown in FIGS. 1A-C, the non-suture soft tissue anchor device 100 comprises a body component 106 configured in an oval shape, such that a middle part 108 tapers to opposing narrow ends 110 to form the oval shape. The body component 106 can be configured in multiple sizes depending on the size of the tendon 102 to be secured. Further, any suitable shape as is known in the art can be utilized for the body component 106, as well, such as square, rectangular, circular, etc.

Furthermore, the body component 106 comprises a top surface 112, a bottom surface 114, and a curved sidewall 116 which runs along the perimeter, defining the oval shape. Further, the top surface 112 and the bottom surface 114 share the curved sidewall 116 extending therebetween. The bottom surface 114 contacts the bone 104 and the top surface 112 is positioned away from the bone 104, when the device 100 is in position.

Additionally, the body component 106 comprises at least two through-holes 118, with one through-hole 118 positioned on each opposing narrow end 110 of the body component 106. The through-holes 118 are sized and shaped to accept a bone screw 120 to secure the device 100 to the bone 104. The bone screws 120 can be any suitable bone screws as is known in the art. The screws 120 can be machined or 3D printed from titanium, stainless steel, PEEK, or PEEK enhanced with hydroxyapatite. The screws 120 can also be locking or non-locking, fixed angle, or variable angle screws that are inserted into the soft tissue anchor device 100.

In one embodiment, the body component 106 is secured without bone screws 120. In this embodiment, a user would insert wires, pins, or staples (not shown) into the two through-holes 118, to secure the device 100 to the bone 104.

Additionally, the non-suture tissue anchor device 100 may be implemented with a range of sizes that facilitate securing the soft tissue 102 to bone 104 in various locations in the human body, such as by way of non-limiting example, the dorsal aspect of the foot, the medial aspect of the foot, the lateral aspect of the foot, the anterior portion of the calcaneus, as needed, as well as multiple portions of the hand. It is contemplated, however, that the overall size of the device 100 is to be selected according to the particular soft tissue 102 that is to be attached. Thus, the device 100 can be scaled up or down based on the size of the soft tissue 102 and the position of the device 100 on the cortical bone 104; and the device 100 acts to secure the soft tissue 102 to the bone 104 without the use of multiple devices 100.

Generally, the device 100 possesses a thickness that extends from the bottom surface 114 to the top surface 112. Generally, the thickness is substantially 1-3 millimeters (mm). It is contemplated, however, that the thickness may be varied according to placement in the foot or hand, and thus the device 100 may be implemented with a wide variety of thicknesses, without limitation.

Furthermore, when the non-suture soft tissue anchor device 100 is implanted to secure the soft tissue 102 to the cortical surface of the bone 104, the top surface 112 of the device 100 is disposed slightly above the cortical bone's surface. In general, the top surface 112 includes a shape configured to approximate the cortical bone's surface. In some embodiments, the shape of the top surface 112 includes a convex curvature that approximates the curvature of the cortical surface of the bone 104. In one embodiment, the top surface 112 may have a flat curvature depending on the curvature of the cortical bone 104 on which it is secured.

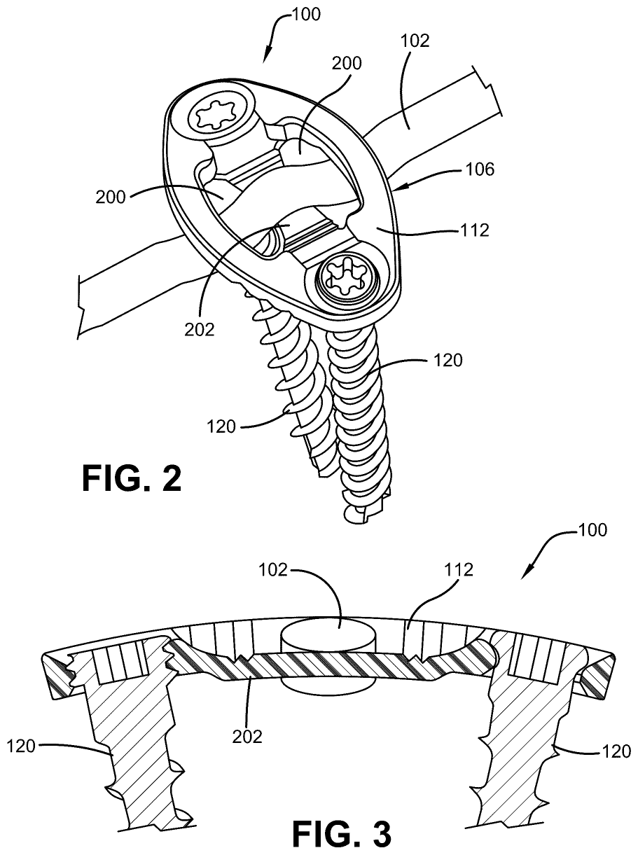

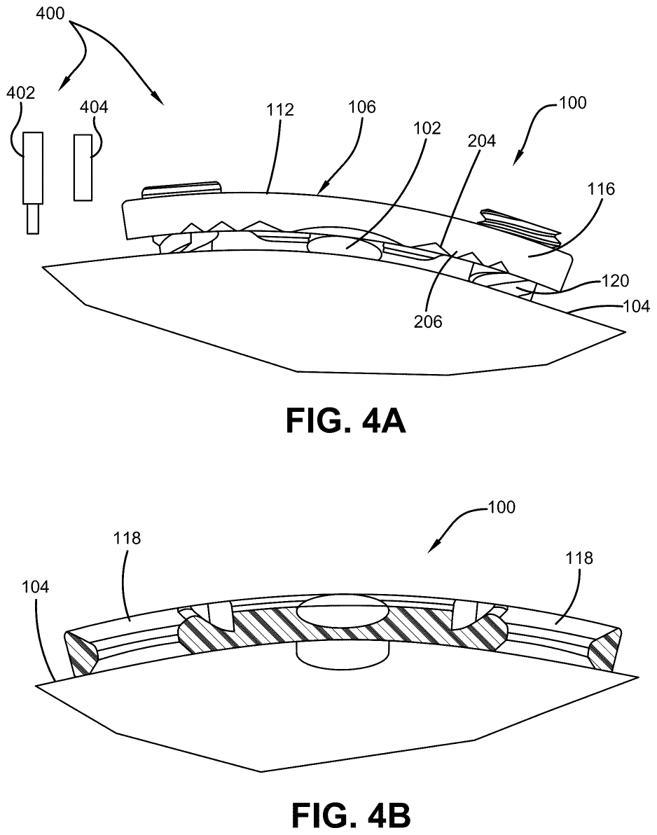

As shown in FIGS. 2-3, the body component 102 comprises two or more slots 200 to accept the soft tissue 102 during use of the device 100. Specifically, the soft tissue 102 is weaved through the slots 200 like a belt buckle to lock the soft tissue 102 in place. The soft tissue 102 is locked in place at a set tension. Thus, the device 100 provides the ability to lock the soft tissue 102 at a specific tension without violating the structural integrity of the soft tissue 102. The slots 200 are configured in an oval shape and span the length of the middle part 108. However, the slots 200 can be configured in any suitable shape and size as is known in the art, as long as they are of the appropriate length and width to accept soft tissue 102. After the soft tissue 102 is fed through the slots 200, the surgeon/user can tension the soft tissue 102 to a desired tension. If the initial tension is later found to be inadequate, the surgeon/user can pull the soft tissue 102 and/or re-position the soft tissue 102 within the slots 200. Thus, the device 100 allows for the re-tensioning of soft tissue 102, multiple times, if needed.

Furthermore, the slots 200 define a moveable lock bar 202, which further locks the soft tissue 102 between the bone 104 and the device 100. The moveable lock bar 202 is configured in a rectangular shape and sits lower than the bottom surface 114 of the body component 106 and contacts the bone 104 when the device 100 is in position. The moveable lock bar 202 is positioned between the two slots 200. In use, the soft tissue 102 is placed under the bottom surface 114 of the body component 106, the soft tissue 102 is then passed up through a first slot 200 and over the center moveable lock bar 202. After the soft tissue 102 is passed over the center moveable lock bar 202, the soft tissue 102 is then threaded through the second (or opposing) slot 200 and pulled out under the opposing bottom surface 114 of the body component 106. Once the soft tissue 102 is completely threaded through the slots 200 and moveable lock bar 202, the surgeon/user can tension the soft tissue 102 to a desired tension. The screws 120 can then be tightened down to pull the device 100 in contact with the bone 104. In tightening the device 100 to the bone 104, the center moveable lock bar 202 will then deform and be pushed away from the bone 104 and will further act to lock the soft tissue 102 into the device 100. Thus, the device 100 allows the surgeon to tension the soft tissue 102 and lock the tissue 102 at a desired tension, without having to move or relocate the body component 106 secured to the bone 104.

Additionally, the bottom surface 114 of the body component 106 comprises a plurality of ridges 204 to further help secure the device 100 to the bone 104. The plurality of ridges 204 contact the bone 104 and/or the soft tissue 102 when the device 100 is in position. The plurality of ridges 204 act to mitigate migration of the soft tissue 102 once in position, securing the device 100 in place. Specifically, the ridges 204 are triangular with the point 206 pushing down toward the bone 104. Further, the ridges 204 are of any suitable length, as long as the length enables pushing of the soft tissue 102 to the bone 104 without causing unnecessary damage to the soft tissue 102.

In one embodiment, the device 100 can be machined, molded or 3D printed. Further, the device 100 may comprise any synthetic or natural homogenous material suitable for implantation into bone 104, including any one or more of collagen, human or animal allograft, silicone, bioglass, collagen, PEEK, polyethylene, titanium, stainless steel, cobalt chrome, and the like, but is typically manufactured of PEEK or PEEK enhanced with hydroxyapatite.

Further, within the body, tendons (i.e., a type of soft tissue 102) typically glide within synovial sheaths (not shown). The sheaths often contain synovial fluid. Synovial fluid lubricates the tendons 102, promoting a smooth glide between the tendons 102 and the sheaths. The synovial fluid is also the primary source of nutrition for the fibrocyte cells of the tendon 102. The non-suture soft tissue anchor device 100 does not act to cover the soft tissue 102 during use, and thus allows synovial fluid to reach the tendon 102 and prevents necrosis of the tendon 102, while speeding the healing process.

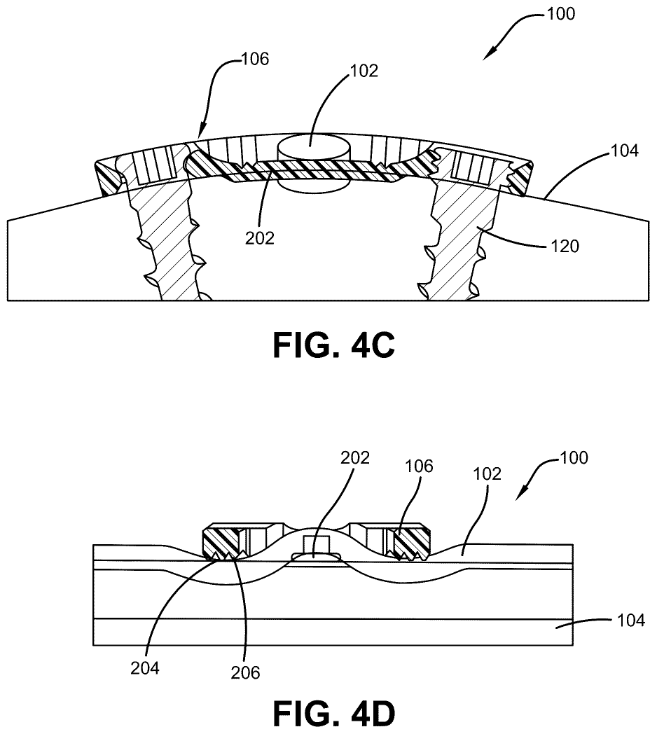

As shown in FIGS. 4A-D, the non-suture soft tissue anchor system 400 for securing soft tissue 102 to bone 104 according to the present disclosure includes the soft tissue anchor device 100, a drill 402, a screw inserter 404, and bone screws 120. As will be appreciated, the non-suture soft tissue anchor system 400 comprises instruments 402 and 404 necessary for securing soft tissue 102 to bone 104 by way of surgery. The sizes of the instruments 402 and 404 comprising the anchor system 400 will depend upon the size of the particular device 100 to be utilized. It is envisioned, therefore, that a surgeon may select the device 100 and a correspondingly sized instrument 402 and 404 based on the location and size of the soft tissue 102 to be secured. The instruments comprising the soft tissue anchor system 400 are not to be limited to the specific instruments, or the sizes and shapes of the instruments disclosed.

A surgical method for securing soft tissue to bone, will now be described. The method utilizes some of the devices, instruments, features, aspects, components and the like described above, and therefore reference will be made to the above-described embodiments, such as the illustrated embodiments presented in the figures and discussed above. However, such references are made for exemplary purposes only and are not intended to limit the surgical method beyond the specifically recited steps. Further, the surgical method may be discussed under the umbrella of particular bones, but such an application is not intended to be limiting and the method described herein may be used or conducted with bone or other tissue not specifically discussed herein without departing from the spirit and scope of the surgical method.

In operation, first, the soft tissue anchor device 100 is placed in the desired position on the cortical bone 104 that is where the soft tissue 102 is needing to be attached to. Next, screw holes are then drilled through the opposing through-holes 118 of the body component 106 via the drill 402. Then, screws 120 are placed through the drill holes and into the bone 104 and tightened via the screw inserter 404. Typically, the screws 120 are torqued into the bone 104 but not sealed completely to the bone 104. Thus, there is a gap left between the body component 106 and the bone 104. Once the body component 106 is in position, the soft tissue 102 is woven through the slots 200 to be secured. Specifically, the soft tissue 102 is placed under the bottom surface 114 of the body component 106, the soft tissue 102 is then passed up through a first slot 200 and over the center moveable lock bar 202. After the soft tissue 102 is passed over the center moveable lock bar 202, the soft tissue 102 is then threaded through the second (or opposing) slot 200 and pulled out under the opposing bottom surface 114 of the body component 106.

In one embodiment, once the soft tissue 102 is completely threaded through the slots 200 and moveable lock bar 202, the surgeon/user can tension the soft tissue 102 to a desired tension. The surgeon/user can pull back and forth on the soft tissue 102 to achieve the desired tension. Once the desired tension is reached, the screws 120 are then tightened down via the screw inserter 404 to pull the soft tissue anchor device 100 in contact with the cortical bone 104. In tightening the device 100 to the bone 104, the center moveable lock bar 202 will then deform and be pushed away from the bone 104 and will further act to lock the soft tissue 102 into the device 100. Thus, the device 100 allows the surgeon to tension the soft tissue 102, re-tension the soft tissue 102, and finally lock the soft tissue 102 at a desired tension, all without having to move or relocate the body component 106 secured to the bone 104.

In general, it is contemplated that the non-suture soft tissue anchor system 400 is to be suitably sterilized for surgeries and packaged into sterilized containers (not shown).

Certain terms are used throughout the following description and claims to refer to particular features or components. As one skilled in the art will appreciate, different users may refer to the same feature or component by different names. This document does not intend to distinguish between components or features that differ in name but not structure or function. As used herein “non-suture soft tissue anchor device”, “soft tissue anchor device”, “anchor device”, and “device” are interchangeable and refer to the non-suture soft tissue anchor device 100 of the present invention.

Notwithstanding the forgoing, the non-suture soft tissue anchor device 100 of the present invention can be of any suitable size and configuration as is known in the art without affecting the overall concept of the invention, provided that it accomplishes the above stated objectives. One of ordinary skill in the art will appreciate that the non-suture soft tissue anchor device 100 as shown in FIGS. 1-4 is for illustrative purposes only, and that many other sizes and shapes of the non-suture soft tissue anchor device 100 are well within the scope of the present disclosure. Although the dimensions of the non-suture soft tissue anchor device 100 are important design parameters for user convenience, the non-suture soft tissue anchor device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A non-suture soft tissue anchor device for securing soft tissue to bone, the non-suture soft tissue anchor device comprising:
a body component configured in an oval shape;
wherein the body component comprises at least two slots positioned in a middle portion of the body component for receiving soft tissue;
wherein the body component comprises at least two through-holes positioned on opposing ends of the body component for receiving bone screws;
wherein soft tissue is woven through the at least two slots and tensioned; and
further wherein the body component is secured via bone screws through the at least two through-holes to bone.

2. The non-suture soft tissue anchor device of claim 1, wherein the body component comprises a top surface, a bottom surface, and a curved sidewall which runs along a perimeter, defining the oval shape, and the curved sidewall extends therebetween the top surface and the bottom surface.

3. The non-suture soft tissue anchor device of claim 2, wherein the bone screws can be machined or 3D printed from titanium, stainless steel, PEEK, or PEEK enhanced with hydroxyapatite.

4. The non-suture soft tissue anchor device of claim 3, wherein the body component is manufactured in a range of sizes.

5. The non-suture soft tissue anchor device of claim 4 wherein the body component possesses a thickness that extends from the bottom surface to the top surface, which is substantially 1-3 millimeters (mm).

6. The non-suture soft tissue anchor device of claim 5 wherein the top surface of the body component comprises a convex curvature that approximates a curvature of a cortical surface of the bone.

7. The non-suture soft tissue anchor device of claim 6, wherein the top surface of the body component comprises a flat curvature that approximates a curvature of a cortical surface of the bone.

8. The non-suture soft tissue anchor device of claim 7, wherein the body component further comprises a moveable lock bar, which is positioned between the at least two slots.

9. The non-suture soft tissue anchor device of claim 8, wherein the moveable lock bar is configured in a rectangular shape and sits lower than the bottom surface of the body component and contacts the bone.

10. The non-suture soft tissue anchor device of claim 9, wherein the soft tissue is placed under the bottom surface of the body component, the soft tissue is then passed up through a first slot and over the moveable lock bar, then the soft tissue is passed over the moveable lock bar and threaded through a second slot and pulled out under an opposing bottom surface of the body component.

11. The non-suture soft tissue anchor device of claim 10, wherein once the soft tissue is completely threaded through the at least two slots and the moveable lock bar, a user can push and pull the soft tissue to set a desired tension.

12. The non-suture soft tissue anchor device of claim 11, wherein the bone screws are tightened down to pull the body component in contact with the bone, and thus the moveable lock bar will deform and be pushed away from the bone to further lock the soft tissue in place.

13. A non-suture soft tissue anchor device for securing soft tissue to bone, the non-suture soft tissue anchor device comprising:
a body component configured in an oval shape and comprising a top surface, a bottom surface, and a curved sidewall which runs along a perimeter, defining the oval shape, and the curved sidewall extends therebetween the top surface and the bottom surface;
wherein the body component comprises at least two slots positioned in a middle portion of the body component for receiving soft tissue;
wherein the body component comprises at least two through-holes positioned on opposing ends of the body component for receiving bone screws;
wherein the body component further comprises a moveable lock bar, which is positioned between the at least two slots;
wherein the moveable lock bar is configured in a rectangular shape and sits lower than the bottom surface of the body component and contacts the bone;
wherein the soft tissue is placed under the bottom surface of the body component, the soft tissue is then passed up through a first slot and over the moveable lock bar, then the soft tissue is passed over the moveable lock bar and threaded through a second slot and pulled out under an opposing bottom surface of the body component;
wherein once the soft tissue is completely threaded through the at least two slots and the moveable lock bar, a user can push and pull the soft tissue to set a desired tension; and
further wherein the bone screws are tightened down to pull the body component in contact with the bone, and thus the moveable lock bar will deform and be pushed away from the bone to further lock the soft tissue in place.

14. The non-suture soft tissue anchor device of claim 13, wherein the bone screws can be machined or 3D printed from titanium, stainless steel, PEEK, or PEEK enhanced with hydroxyapatite.

15. The non-suture soft tissue anchor device of claim 13, wherein the body component is manufactured in a range of sizes.

16. The non-suture soft tissue anchor device of claim 13, wherein the body component possesses a thickness that extends from the bottom surface to the top surface, which is substantially 1-3 millimeters (mm).

17. The non-suture soft tissue anchor device of claim 13, wherein the bottom surface of the body component comprises a plurality of ridges to contact the bone and the soft tissue.

18. The non-suture soft tissue anchor device of claim 17, wherein the plurality of ridges are each triangular with a point for pushing down and act to mitigate migration of the soft tissue once in position.

19. The non-suture soft tissue anchor device of claim 13, wherein the body component can be machined, molded or 3D printed of PEEK or PEEK enhanced with hydroxyapatite.

20. A non-suture soft tissue anchor system for securing soft tissue to bone, the non-suture soft tissue anchor device comprising:
a non-suture soft tissue anchor device;
a drill;
a screw inserter; and
a plurality of bone screws.
